(19) United States
(12) Patent Application Publication
(10) Pub. No.: US 2006/0142825 A1
(43) Pub. Date: Jun. 29, 2006

(54) Patient warming system

(76) Inventor: Colin Dunlop, New South Wales (AU)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/538,303

(22) PCT Filed: Dec. 9, 2003
(86) PCT No.: PCT/AU03/01626

§ 371 Date: Dec. 2, 2005

(30) Foreign Application Priority Data
Dec. 9, 2002 (AU) 2002953185

(51) Int. Cl. A61F 7/00

(52) U.S. Cl. 607/104

(57) ABSTRACT

The present invention relates to a patient warming system and in particular a patient warming blanket for warming patients undergoing medical care. The patient warming blanket is particularly for use in veterinary medicine. The warming blanket includes a porous surface from which warmed air can escape over the entire porous surface, evenly warming the patient.

### FIELD OF THE INVENTION

The present invention relates to a warming system for patient care and, particularly, but not exclusively, to a warming system for use in veterinary care.

### BACKGROUND OF THE INVENTION

There are many circumstances in human and animal medicine where it is necessary to keep a patient warm to, for example, prevent or treat hypothermia.

In human medicine, it is known to provide patient warming systems which include a patient warming blanket and a heating unit. The patient warming blanket includes two layers which are bonded or stitched together at a seam and are otherwise separable from each other to form a hollow space within the blanket when warm air is pumped from the heating unit via a delivery tube in between the two layers. One of the layers contains a plurality of air holes which allow the pumped warm air to escape from the blanket. In operation, the patient is wrapped or covered in the warming blanket with the layer with the holes next to the patient. Warm air is pumped in from the heating unit and escapes from the air holes on the inside layer of the blanket and keeps the patient warm.

These patient warming systems are designed for use in human medicine only, for the prevention and treatment of hypothermia during anaesthesia and critical care.

There is, however, a similar need for a patient warming system in veterinary care. Presently, similar warming systems are used as those designed for human patients. There are a number of problems associated with the use of the human patient warming system in veterinary care, however.

Small animals have a relatively large surface area to volume ratio, which makes them particularly susceptible to hypothermia. The applicants have found that using a conventional human warming system to maintain the body temperature of a relatively small animal can actually result in cooling of the animal (which can lead to death). This occurs, we believe, because the air flow is delivered to the patient by individual, discreet holes in the inner layer of the warming blanket. In a patient with relatively large surface area to volume, delivery of air from an air hole, so that the air is moving relatively rapidly, can cause the patient to chill, as the air takes away more heat from the surface of the patient than it delivers. Obviously, this is very dangerous in a critical care situation.

Another problem is that the heating unit used in the human systems typically only heats to a temperature of 43° C. Animals have a range of body temperatures and in many circumstances a system which provides heated air at a maximum of 43° C. is not sufficient.

Another problem which relates to animals, which in veterinary situations are often smaller and sometimes much smaller than human beings, is that the human patient warming blankets are relatively large, and a small animal placed under one of these will not be adjacent sufficient air holes to provide sufficient warm air to maintain the animal's temperature.

Further, in surgery and other circumstances where sterile conditions are required, having air blown at relatively high velocity through a small hole can result in contamination of the site eg. the surgical site, via substances blown onto the surgical site by air from the air holes.

### SUMMARY OF THE INVENTION

In accordance with a first aspect, the present invention provides a surgical warming blanket arranged for use during surgery on a patient and comprising at least two layers capable of forming a hollow air space between them for receiving warmed air from a heating unit, the two layers and air space being arranged in operation to form a substantially tubular arrangement at least partially surrounding a patient receiving space, whereby when warm air is passed into the air space it is delivered to the patient receiving space via the blanket, to maintain warm air within the patient receiving space, the patient receiving space receiving the patients body and allowing access to the patients body for surgery without disturbing the blanket.

In one embodiment, at least one of the two layers has a proportion of its surface formed of porous material so that warmed air may escape via the porous material into the patient receiving space.

The pervious material is adjacent to, in use, a patient receiving treatment. Delivering heat spread over the surface of the porous material advantageously has the effect of evenly warming the patient without forming relatively high velocity streams of air (as in the prior art blanket where the air is delivered via discreet holes). Animals, therefore, and in particular small animals, are not at risk of being cooled by relatively high velocity air streams. In one embodiment, a substantial proportion of the surface of the one layer is of porous material. Preferably, a majority of the surface of the one layer is of porous material. In operation, warm area is advantageously delivered at relatively low velocity over the proportion of the surface of the one layer.

Preferably, the blanket is designed not to cover the animal patient, but instead to provide a patient receiving area in which the patient lies surrounded at least on three sides by a tube formed by the blanket when air is pumped into the air space. In this embodiment, at least the sides of the tube facing inwards towards the patient are of the porous material. This has the effect of passing warm air over the patient within the space, so no matter how large the patient, the air in the space will be kept at substantially the same temperature.

Preferably, the surface of the blanket is fluid repellent, so that any liquid contamination rolls off the blanket.

In an alternative embodiment, the entire blanket may be made of porous material so that warmed air is delivered over the entire surface of the blanket that is exposed. The unexposed surface of the blanket e.g. facing down on a bench, may not deliver air. The exposed surface, however, including the surface which may be adjacent to patient in operation, will deliver air. This saves cost in manufacture of the blanket as it is only necessary to manufacture the blanket from one type of material. This can be significant, as in the majority of cases these blankets are intended to be disposable after one use.

In an alternative embodiment, the entire blanket may be made of porous material so that warmed air is delivered over the entire surface of the blanket that is exposed. The unexposed surface of the blanket e.g. facing down on a bench, may not deliver air. The exposed surface, however, including the surface which may be adjacent to patient in operation, will deliver air. This saves cost in manufacture of the blanket as it is only necessary to manufacture the blanket from one type of material. This can be significant, as in the majority of cases these blankets are intended to be disposable after one use.

One other problem with the conventional human patient warming systems is that it has been known for carers to direct heat directly from the heating unit via a delivery tube directly onto the patient. This can cause burning, particularly in small animals, and is not something that should occur.

In accordance with a third aspect, the present invention provides a heating unit for a patient warming system, the heating unit including a delivery port for delivering warmed air to a patient warming blanket, and a feedback means for determining whether a patient warming blanket is attached and being responsive to a determination that the patient warming blanket is not attached, to disable delivery of warmed air via the port.

Preferably, the feedback means comprises a pressure sensor, for sensing back pressure on the air delivery port. When a blanket is attached, there will be a certain amount of back pressure on the delivery port, so that when this back pressure is detected, air may delivered.

Preferably, the heating unit is arranged to heat air to a range of temperatures, preferably up to 46° C.

In accordance with a fourth aspect, there is provided a heating system comprising a patient warming blanket in accordance with the first aspect of the present invention and a heating unit in accordance with the third aspect of the present invention.

In accordance with a fifth aspect, the present invention provides a method of warming a patient during surgery, comprising the steps of receiving the patient within a patient receiving space within which the patients body is accessible for surgery, and passing warmed air into the patient receiving space to keep the patient warm.

### BRIEF DESCRIPTION OF THE DRAWINGS

FIG. 1 is a plan view of a patient warming blanket in accordance with one embodiment of the present invention, shown connected to a heating unit in accordance with one embodiment of the present invention;

FIG. 2 is a cross sectional view on line XX of FIG. 1;

FIG. 3 is a plan view of a patient warming blanket in accordance with a further embodiment of the present invention; and

FIG. 4 is a view from the front of the embodiment of FIG. 3.

### DESCRIPTION OF PREFERRED EMBODIMENT

With reference to the figures, a patient warming system in accordance with an embodiment of the present invention is illustrated, particularly being designed for use in veterinary medicine. The patient warming system comprises a heating unit 1 (to be described in more detail later) and a patient warming blanket 2.

The patient warming blanket 2 includes first 3 and second 4 layers of material which form a hollow air space 5 between them. In this embodiment, when the warming blanket 2 is not being used, it will lie substantially flat as no air is being pumped into the air space 5. In use, however, when air is being pumped into the air space 5, the blanket “inflates” to give the profile shown in the cross-section of FIG. 2.

The first layer 3 is substantially non porous to air. The second layer 4, however, is made of porous material and is substantially porous over its entire surface area. Warm air pumped into the hollow air space 5, therefore, escapes via the entire surface of the second layer 4.

The warming blanket 2 may be made of any appropriate material and in this embodiment is made from polyester. The second surface 4 being of porous polyester.

The arrangement of the first 3 and second layers 4 in operation in this embodiment forms a tubular arrangement which extends about three sides of a patient receiving space 6. In this embodiment, a continuation 7 of the first layer 3 provides a blanket base on which the patient may lie.

In operation, warmed air is provided from the heating unit 1 via a flexible heat delivery tube 8 into a port 9 to the interior space 5 of the blanket. The warmed air inflates the blanket to give the profile illustrated in FIG. 2. The patient is positioned within the patient receiving space 6. Warm air escapes via the porous second layer 4 into the patient receiving space maintaining the patient receiving space 6 at a substantially even temperature. The shape of the blanket maximises the convective surface area for heating.

The material of the warming blanket 2 is treated to be fluid repellent, so that any liquid contamination rolls off the blanket.

In an alternative embodiment, the blanket may consist of the same main material over all of its surface. Warmed air is therefore delivered over all of the exposed surface of the blanket. This blanket may be cheaper to make.

The heating unit 1 includes a feedback means which in this embodiment is a pressure sensor. The pressure sensor is arranged to sense a certain amount of back pressure on a delivery port 10 of the heating unit which delivers warmed air to the delivery tube 8. The existence of this back pressure implies that a warming blanket 2 is attached to the delivery tube 8. If the back pressure signal is not received by the pressure sensor, then delivery of warmed air 10 via the port is disabled. This prevents any operative attempting to provide warmed air directly to a patient via the delivery tube 8, without using a warming blanket.

The heating unit 1 includes control and selection means 12, 13, 14 that enables a selection of plurality of temperatures for the warmed air, and in this embodiment warmed air can be delivered at temperatures of 34, 37, 40, 43 or 46 degrees Centigrade.

The heating unit 1 is based on a conventional heating unit, but adapted to deliver the above temperatures. A further adaptation is the addition of the pressure sensor and feedback to temperature control circuitry (not shown) to switch off the delivery of warmed air if a back pressure is not sensed (implying that the warming blanket 2 is not attached to the delivery tube 8).

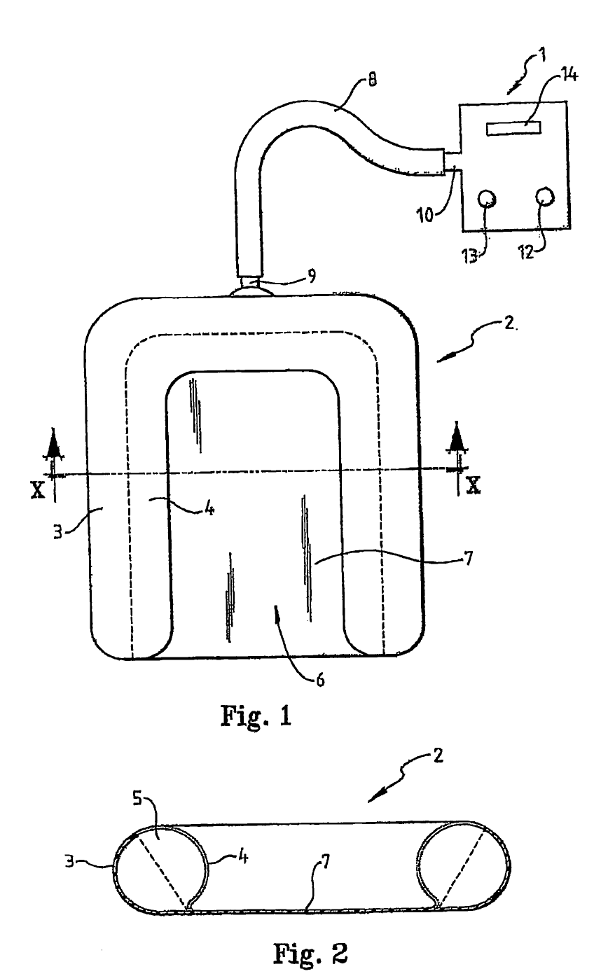

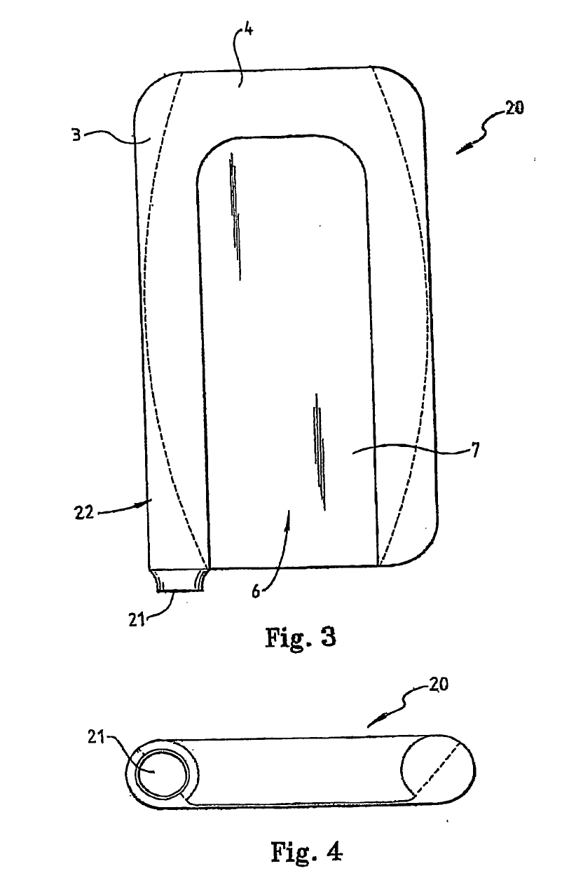

FIG. 3 and FIG. 4 show an alternative embodiment of the patient warming blanket. The alternative patient warming blanket 20 comprises an air inlet 21 which is on one “leg” 22 of the blanket. Otherwise, the blanket is of similar construction to the patient warming blanket of FIGS. 1 and 2. Similar reference numerals have been used for similar components as the embodiment of FIGS. 1 and 2.

In the above-described embodiment the patient warming blanket will be appropriately dimensioned for veterinary care. Example dimensions include 560 mm width, 1110 mm length, with width of each of the arms when inflated being 110 mm. Note that these dimensions are examples only and, the present invention is not limited to these dimensions.

While the above description refers to application of the warming system with animal patients, the system of the present invention is not limited to use with animal patients and can be used with human patients eg. small human patients.

Modifications and variations as would be apparent to a skilled addressee are deemed to be within the scope of the present invention.

1-14. (canceled)

15. A surgical warming blanket arranged for use during surgery on a patient and comprising at least two layers capable of forming a hollow air space between the two layers for receiving warmed air from a heating unit, the two layers and air space being arranged in operation to form a substantially tubular arrangement at least partially surrounding a patient receiving space, whereby when warm air is passed into the air space the warm air is delivered to the patient receiving space via the blanket, to maintain warm air within the patient receiving space, the patient receiving space receiving the patient's body and allowing access to the patient's body for surgery without disturbing the blanket.

16. The surgical warming blanket in accordance with claim 15, wherein the tubular arrangement surrounds the patient receiving space on three sides.

17. The surgical warming blanket in accordance with claim 15, wherein one of the two layers of the blanket has a portion of its surface formed of pervious material so that the warmed air is delivered to the patient receiving space via the pervious material.

18. The surgical warming blanket in accordance with claim 15, wherein the surface of the blanket is arranged to be fluid repellent, so that liquid contamination is repelled by the surface of the blanket.

19. The surgical warming blanket in according to claim 15, wherein the surgical warming blanket is sized and shaped so that the patient receiving space is arranged to receive an animal.

20. The surgical warming blanket in accordance with claim 19, wherein the surgical warming blanket is shaped and sized so that the patient receiving space is arranged to receive a large animal, such as a large dog.

21. The surgical warming blanket in accordance with claim 15, the surgical warming blanket is sized and shaped so that the patient receiving space can receive a human adult.

22. A heating unit for a patient warming system, the heating unit including a delivery port for delivering warmed air to a patient warming blanket, and a feedback means for determining whether a patient warming blanket is attached and responsive to a determination that the patient warming blanket is not attached, to disable delivery of warmed air via the port.

23. The heating unit in accordance with claim 22, wherein the feedback means includes a pressure sensor for sensing back pressure on the air delivery port.

24. The heating unit in accordance with claim 22, the heating unit being arranged to heat the air to a range of temperatures.

25. The heating unit in accordance with claim 24, being arranged to heat air up to 46° C.

26. The heating unit in accordance with claim 22, wherein in combination with the surgical warming blanket arranged for use during surgery on a patient, the blanket having at least two layers capable of forming a hollow air space between the two layers for receiving warmed air from the heating unit, the two layers and air space being arranged in operation to form a substantially tubular arrangement at least partially surrounding a patient receiving space, whereby when the warm air is passed into the air space the warm air is delivered to the patient receiving space via the blanket, to maintain warm air within the patient receiving space, the patient receiving space receiving the patient's body and allowing access to the patient's body for surgery without disturbing the blanket.

27. A method of warming a patient during surgery, comprising the steps of receiving the patient within a patient receiving space within which the patient's body is accessible for surgery, and passing warmed air into a patient receiving space to keep the patient warm.

28. The method in accordance with claim 27, further comprising the step of utilizing a surgical warming blanket arranged for use during surgery on a patient, the warming blanket comprising at least two layers capable of forming a hollow air space between them for receiving warmed air from a heating unit, the two layers and air space being arranged in operation to form a substantially tubular arrangement at least partially surrounding a patient receiving space, whereby when warm air is passed into the air space the warm air is delivered to the patient receiving space via the blanket, to maintain warm air within the patient receiving space, the patient receiving space receiving the patients body and allowing access to the patients body for surgery without disturbing the blanket to form the patient receiving space and deliver the warmed air thereto.
